(19) United States
(12) Patent Application Publication
(10) Pub. No.: US 2016/0022754 A1
(43) Pub. Date: Jan. 28, 2016

(54) Supplement Composition and Methods of Use

(71) Applicant: O3 Animal Health, LLC, Cleveland, MS (US)

(72) Inventor: Murray Fulgham, Cleveland, MS (US)

(21) Appl. No.: 14/874,649

(22) Filed: Oct. 5, 2015

Related U.S. Application Data
(63) Continuation-in-part of application No. 14/228,242, filed on Mar. 27, 2014.
Application No. 14/228,242 is a continuation-in-part of application No. 13/969,083, filed on Aug. 16, 2013, now Pat. No. 8,716,331.
Application No. 13/969,083 is a continuation of application No. 13/012,255, filed on Jan. 24, 2011, now Pat. No. 8,536,220.
Application No. 13/012,255 is a continuation-in-part of application No. 14/228,234, filed on Mar. 27, 2014.
Application No. 14/228,234 is a division of application No. 13/969,057, filed on Aug. 16, 2013, now Pat. No. 8,716,330.
Application No. 13/969,057 is a continuation-in-part of application No. 13/012,255, filed on Jan. 24, 2011, now Pat. No. 8,536,220.
(60) Provisional application No. 61/298,260, filed on Jan. 26, 2010.
(60) Provisional application No. 61/298,260, filed on Jan. 26, 2010.

(51) Int. Cl. A61K 36/48; A23K 1/18; A23K 1/16; A61K 36/45; A61K 35/60

(52) U.S. Cl. CPC A61K 36/48; A61K 36/45; A61K 35/60; A23K 1/164; A23K 1/1806

(57) ABSTRACT

The present disclosure provides a supplement composition for an animal comprising: cranberry oil in the volume range of 10-30%, 20-40% soybean oil in the volume range of and fish oil in the volume range of 30-70%.

### CROSS-REFERENCES TO RELATED APPLICATIONS

This application is a continuation-in-part of pending U.S. application Ser. No. 14/228,242 filed Mar. 27, 2014, which in turn was a continuation-in-part of U.S. application Ser. No. 13/969,083 filed on Aug. 16, 2013 and now U.S. Pat. No. 8,716,331, which was a continuation of U.S. application Ser. No. 13/012,255 filed on Jan. 24, 2011 and now U.S. Pat. No. 8,536,220, which claimed priority to U.S. provisional application No. 61/298,260, all of which are hereby incorporated by reference in their entirety.

This application is also a continuation-in-part of pending U.S. application Ser. No. 14/228,234 filed Mar. 27, 2014, which was a divisional of U.S. application Ser. No. 13/969,057 and now U.S. Pat. No. 8,716,330 which was a continuation-in-part of U.S. application Ser. No. 13/012,255 filed on Jan. 24, 2011 and now U.S. Pat. No. 8,536,220, which claimed priority to U.S. provisional application No. 61/298,260, all of which are hereby incorporated by reference in their entirety.

### FIELD OF THE INVENTION

The present invention is related to an animal nutritional supplement and, more particularly to a method of using a nutritional supplement to alleviate chronic or acute conditions in domestic animals such as equines, canines and felines.

### BACKGROUND OF THE INVENTION

Equines are known to suffer from a number of conditions related to vitamin and minerals deficiencies due to poor quality forage or hay, chronic colic, chronic diarrhea, or anorexia resulting from dental disease. In addition, there may also be disturbances in absorption as the result of liver or biliary tract disease, hypothyroidism, anemia and other pathological conditions of the digestive system and related organs. Numerous equine supplements are currently on the market. Some of these supplements include various Omega 3 containing products. The Omega 3 oils in current production are from a variety of sources, strengths, and types of Omega 3 that supply EPA, DHA or ALA for supplementation of the equine diet. Some are from marine oils such as fish oil which contain different amounts of DHA and EPA, the long chain Omega 3s, depending on which fish and which part of the ocean they came from. Some come from vegetable oils such as soybean oil, canola oil, or flax seed, all of which contain ALA Omega 3, the parent chain of Omega 3. However, the equine diet in the wild provides a balance of Omega 3, Omega 6 and Omega 9. Domestic animal feeds may disturb this balance and result in chronic or acute conditions. Other animals, such as canines and felines may also benefit from the same supplements as equines for a similar number of reasons.

### SUMMARY OF THE INVENTION

Thus, it is a purpose of the present invention to provide a nutritional supplement for alleviating various chronic or acute conditions in domestic animals associated with an imbalance of Omega 3, Omega 6 and Omega 9. It is also a purpose of the present invention to provide a method of use for the nutritional supplement, of the present invention, to alleviate chronic or acute conditions in domestic animals.

in accordance with a preferred embodiment of the invention, an equine nutritional supplement for improving omega balance in domestic animals is made of: a balance of Omega 3, Omega 6, and Omega 9 plus other nutrients, including from the acai berry, in expeller pressed soybean oil. More specifically, this composition is made of: linolenic expeller pressed soybean oil in a volume range of 65%-85%, Omega 3 fish oil in the range of 15%-35%, and 1%-20% alpha-tocopherol (natural vitamin E). This invention further provides a method for alleviating chronic or acute conditions in animals by the method of administering a nutritional supplement to the animal.

This invention more specially provides a method for alleviating anhidrosis in an equine, especially when the equine has been non-responsive to other treatment methods and compositions. This method involves administering a nutritional supplement composition made of 3:1 omega 6 to omega 3, 12,000 IU per serving of alpha-tocopherol, and 7,5% to 15% (total by weight) of acai berry.

In other embodiments, this invention relates to an animal nutritional supplement for improving the health of animals that either consists of consists essentially of or comprises cranberry oil, soybean oil and fish oil, The nutritional supplement may be especially beneficial for horses or other equines. In one embodiment, the supplement may consist of, consist essentially of or comprise 10-30% cranberry oil, 20-40% soybean oil and 30-70% 18/12 fish oil. In a specific embodiment, the nutritional supplement consists of, consists essentially of or comprises 20% expeller cold pressed cranberry oil, 25% expeller cold pressed soybean oil and 55% 18/12 fish oil.

in yet other embodiments, this invention relates to a method of treating illnesses in animals, such as equines, canines and felines by providing the animal the animal supplement composition disclosed herein. In one embodiment, the illness is a cancer such as hemangiosarcoma.

### DETAILED DESCRIPTION OF THE INVENTION

The present invention relates to a nutritional supplement to balance the distribution of Omega 3s, Omega 6s, and Omega 9 in the diet of a domestic animal. The present invention relates to a composition made of: linolenic expeller pressed soybean oil in the range of 65%-85%, Omega 3 fish oil in the range of 15%-35%, and 1%-20% alpha-tocopherol (natural vitamin E). In the preferred embodiment, the fish oil is 18/12. More specifically, the parent chain (ALA) of Omega 3s from natural expeller pressed soy oil and the long chain Omega 3s (DHA and EPA) from natural Omega 3 fish oil and natural alpha-tocopherol (vitamin E) are provided, The alpha-tocopherol has a dual role in providing the needed additional vitamin E and acting as a preservative for this composition, The alpha-tocopherol is in a concentration from between 3,000 to 28,000 IU per serving.

Linolenic expeller pressed soybean oil is made by mechanical (expeller) extraction. During mechanical extraction, an expeller press crushes the soybeans to extract the oil. This pressing is done under intense pressure, and raises the temperature of the oil to 185 to 200° F. (85-93.3° C.). Typically, the soybeans are heated up to 250° F. (120° C.) before being placed in the expeller. Alternatively, the linolenic expeller pressed soybean oil can be cold pressed using filtration and distilled water to filter the oil.

The linolenic expeller pressed soybean oil is admixed with the fish oil and alpha-tocopherol in the volume range of 65%-85%, Omega 3 (18/12) fish oil in the range of 15%-35%, and 1%-20% alpha-tocopherol (natural vitamin E) to form the supplement composition of the present invention.

Bulk Fish Oil comes in two general varieties: 18% EPA, 12% DHA and 30% DHA. Both refer to the general level of omega-3 in the oil. In 18/12, the levels of EPA and DHA are set in the specifications to 18% EPA and 12% DHA. In what is generally considered a lower grade (30% fish oil) the levels of EPA and DHA are not specified and therefore, depend on seasonal variations.

Cranberry oil contains numerous antioxidants and other beneficial components. The antioxidants and beneficial components are often much more concentrated in cranberry oil than other sources. For example, cranberry oil contains a proper balance of omega 3 and 6 fatty acids, natural vitamin e (and other related compounds), alpha and gamma tocopherols, beta-sitosterol and phosphatidylcholine. The cranberry oil may be produced via cold expeller extraction wherein an expeller mechanically crushes the cranberries, or cranberry seeds, releasing the oil therefrom. In some embodiments, the cold expeller extraction involves squeezing the cranberry seeds at temperatures not exceeding 140 degrees Fahrenheit. The cold expeller extraction process helps prevent degradation of the beneficial nutrients found in the cranberry oil.

The animal nutritional supplement can be used for improving or alleviating various chronic or acute conditions in equines, felines or canines associated with an imbalance of Omega 3, Omega 6 and Omega 9. The use of the supplement composition has been show to provide: a healthy digestive system, reduces inflammation in the joints, blocks lactic acid buildup in muscles, increases blood now to all organs and joints, promotes beautiful hair coats and healthy hooves, builds a strong immune system and promotes a healthy reproductive system. The serving size of the present composition for an average horse is 4 ounces daily (1 oz. per 300 lbs.). It is dispensed directly onto feed. If the horse is fed twice daily the dosage can be applied either in one feeding or divided in two and given at both feedings. Serving size can be increased for horses with specific needs or problems. For example, it is recommended to double the dosage a. few days prior to competition for show horses to enhance coat bloom, respiration, and recovery from the extra work and stress of the show. The composition can be formulated as an equine top dress liquid mixture by packaging the liquid product in a gallon container. In one embodiment, the composition is made from ingredients that have been produced using methods of organic farming or may be certified as being organic.

In an alternative embodiment, the supplement composition can be provided as an additive to an equine feed. In another embodiment, the supplement composition can he prepared as a solid supplement. In this embodiment, the supplement composition can be blended with inactive ingredients such as: natural soy lecithin, apple pectin, calcium carbonate, dicalcium phosphate, citric acid, flavoring, anise oil, primary yeast dehydrated, silicon dioxide, yeast extract, yeast fermentation solubles, vegetable stearate, and lignin sulfate. The inactive ingredients can be added to the extent they do not change the fundamental properties of the supplement composition. The solid supplement composition can be formulated into pellets according to techniques known to those skilled in the art.

In an alternative embodiment, a nutritional supplement composition is made of linolenic expeller pressed soybean oil to Omega 3 fish oil in a ratio of 3:1 and alpha-tocopherol (natural vitamin E). Sufficient alpha-tocophero I is added to the mixture to provide 12,000 IU per serving of alpha-tocopherol. This nutritional supplement composition can be used to treat anhidrosis in equines. Equine anhidrosis is the inability in a horse to sweat due to intolerance to heat and humidity. It is estimated that 20%-30% of horses in southern regions suffer sweat disorders (anhidrosis). Horses raised in cool climates and later transferred to very warm southern or tropical areas are prone to this illness.

In yet another alternative embodiment, a nutritional supplement composition is made of linolenic expeller pressed soybean oil, Omega 3 fish oil, and alpha-tocopherol (natural vitamin E) as described in the various embodiments above, with the addition of 7.5% to 15% by weight of acai berry, The acai berry may be from any source, but preferably the acai berry is a freeze-dried powder from an organic source. in the preferred formulation, the composition is made with linolenic expeller pressed soybean oil in the volume range of 65%-85%, Omega 3 fish oil 15%-35%, 1%-20% alpha-tocopherol, The preferred formulation also contains two (2) cups of freeze-dried organic acai berry powder per gallon of the soybean oil, Omega 3 fish oil, and alpha-tocopherol mixture. Other formulations using the teachings above may also be used with the acai berry composition.

The acai berry composition should be mixed well before being applied as a top dressing. No emulsifier is necessary, but any acceptable emulsifier known in the nutritional supplement field may be used. The acai berry composition serving size for an average horse is 4 ounces daily (1 oz. per 300 lbs.). It may be dispensed directly onto feed as a top dressing composition. If the horse is fed twice daily, the dosage can be applied either in one feeding or divided in two and given at both feedings. Serving size can be increased for horses with specific needs or problems, or as a loading regimen by giving the horse three times the daily serving size for the first 1-3 days.

In a specific embodiment, the animal nutritional supplement either consists of, consists essentially of or comprises cranberry oil, soybean oil and fish oil. In one embodiment, the supplement may consist of, consist essentially of or comprise 10-30% cranberry oil, 20-40% soybean oil and 30-70% 18/12 fish oil. In a specific embodiment, the supplement consists of consists essentially of or comprises 20% expeller cold pressed cranberry oil, 25% expeller pressed soy oil and 55% 18/12 fish oil. In one embodiment, the cranberry oil is produced from cranberry seeds contained in the pulp of cranberries that have been crushed previously. The animal nutritional supplement with cranberry oil may be provided to an animal, such as an equine, canine or feline, to treat an illness. The illnesses range from cancer, such as hemangiosarcoma to Cushing's disease to anihydrosis. In one particular embodiment, the animal being treated is a canine and the disease is cancer. In another particular embodiment, the animal being treated is an equine and the disease is either equine's Cushing's disease or anihydrosis. Equine Cushing's disease is one of the most common hormonal disorders that occurs in horses. It is normally due to a tumor, or an increase of cells in the region of the pituitary gland. The tumor or increased number of cells results in the increased production of the stress hormone Cortisol, or mimicking cortisol-like compounds by the adrenal glands which are located near the horses' kidneys. It is believed that the feeding of the supplement described herein may alleviate the symptoms of Equine Cushing's disease in some equines.

### EXAMPLE 1

Equine A, a 2 yr. old western pleasure horse, received four oz. of the composition on a daily basis for eight weeks. After receiving the supplement, it was observed that equine A was quieter and more focused on his training than ever before. The weight gain and bloom within the first month were incredible. Equine B, a young horse with early onset of arthritis, received the composition for its anti-inflarnmatory benefits. It was reported by the attending veterinarian, that the product added weight and bloom on horses quickly and did not affect their energy levels, in contrast to prior supplements.

### EXAMPLE 2

In August, 2010, Equine C could barely stand due to pain. The horse was diagnosed with navicular syndrome, i.e., heel soreness or lameness. With this condition, some horses may be sound with large structural navicular changes whereas others may be extremely lame with minimal radiographic changes. The most commonly seen changes are enlarged blood vessel channels, “lollipop lesions”, spurring, tiny fractures off the navicular edge, cystic or lytic areas within the bone, and erosion of the contact area between the navicular bone and deep digital flexor tendon. Equine C was treated with four oz. of the composition on a daily basis for 12 weeks crossed with the Acuscope/Myoscope treatments. Equine C has been pain free for over one year without injections or other interventions.

### EXAMPLE 3

Four oz. of the alternate composition of this invention was given on a daily basis to Equine group D for anhidrosis for 60 days. An immediate improvement in the ability to sweat was observed. Additionally, the overall condition of Equine group D improved within a few short weeks. The improvements in condition included reduced swelling, the ability to hold their weight better and their hooves and hair coats seemed to have improved as well.

### EXAMPLE 4

Canine A was misdiagnosed with allergies in early June of 2010, and the treatment of antibiotics and steroids sent her into a severe downward spiral. Her condition was diagnosed as Demodex mange, which all puppies carry from their mothers at birth. A healthy immune system keeps puppies from having trouble with these mites. Her immune system was basically shut down from the treatment, Her condition worsened. Her skin was red, and hair was falling out. Canine A was treated with IMMUNOPLEX®, derma support herbal remedies and the present composition at 0.5 ounce per day. Within a week, whiskers of hair appeared and her hair and skin were restored to normal. Canine A has received the treatment continuously since to maintain healthy skin and coat.

### EXAMPLE 5

Equine E was a bottle fed horse experiencing significant trouble with gaining and maintaining weight. Equine E's regular feed was supplemented with about 1 oz. of the composition on a daily basis for one week, then increased dosage to 4 oz. of the composition per day. Within three days, Equine E's coat showed signs of improvement in coat bloom. After the initial week, Equine E began to increase weight gain, which he was able to maintain unlike before using the composition despite decreasing his rations. Equine F's muscle tone has also shown dramatic improvement, which demonstrates that the composition is not leading to “bad” weight gain in the horse.

### EXAMPLE 6

Equine F, a three year old gelding, was experiencing locking up in his stifle joints. Stifle blistering was performed without success. Before resorting to surgery, Equine F was put on a regimen of 3 oz. of the composition three times per day for 15 days. After 15 days of the supplement composition, Equine F is no longer experiencing locking up. Equine F also was suffering from colic, and while on the same regimen showed faster recovery from the digestive blockage (colic). Equine F continues to be colic free and no longer has issues with stifle joint lock up.

### EXAMPLE 7

Equine G, a mare, was suffering from a hock injury that caused considerable lameness, which kept her from being shown. The mare has been treated with four oz. of the composition daily for over three months. The swelling in Equine G's hind leg has completely decreased after being put on the daily regimen of the composition, and her step and reach continues to improve such that her owner is considering showing the horse in the near future.

### EXAMPLE 8

Equine H is a western pleasure horse that had been experiencing anhidrosis. Equine H was treated with four oz. of the anhidrosis formula of the composition daily for one month, then given the supplement composition at the same dosage regimen for five months. Equine H's anhidrosis quickly was alleviated, and also showed great improvement in coat bloom and weight gain. The rations for Equine H were decreased due to the maintenance of the weight gain. Equine H also experienced dramatic improvements in hoof health, no longer showing chips and growing faster.

### EXAMPLE 9

Equine group I is a group of horses that were suffering from joint swelling/pain. Each was given four oz. of the composition added to their feed daily for at least two months. All were reported to have experienced and maintained decreased swelling and pain in their joints, in addition to decreased muscle soreness after working.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### EXAMPLE 10

Equine group J is a group of Thoroughbred horses that were suffering from anhidrosis. They did not respond to the formulations disclosed that did not contain acai berry. However, after receiving the acai berry formulation containing 2 cups of freeze dried organic acai berry powder for as few as one day began to sweat normally.

### EXAMPLE 11

Canine B, of approximately 10 years of age, was diagnosed with advanced hemangiosarcoma with a poor prognosis. The animal nutritional supplement of the present invention consisting of, consisting essentially of or comprising 10-30% cranberry oil, 20-40% soybean oil and 30-70% 18/12 fish oil was fed to the canine as one (1) fluid ounce (1 oz) top dressed over the canine's normal food over a period of months the canine's prognosis and health generally improved and the canine has lived far beyond the time frame provided with the initial diagnosis of the cancer. Additionally, it is believed that provision of the nutritional supplement disclosed herein to canines (and perhaps other animals) will alleviate or reduce the stresses experienced by canines during travel, including without limitation gastrointestinal issues.

### Benefits Offered by the Nutritional Supplement Disclosed Herein

Feeding of the nutritional supplement disclosed herein offers many health benefits to animals, including without limitation the benefits discussed below:

Reduced Tissue and Joint Inflammation:

Prolonged exposure to inflammation perpetuates the cycle of cartilage destruction within the joint and can lead to arthritic conditions. Deterioration of cartilage is often accompanied by changes in the bone and soft tissue. Once inside the body, omega-3 and omega-6 fatty acids have hormone-like properties. With the addition of omega-3 fatty acids to the diet, particularly the long chain fatty acids, eicosapentaenoic acid (EPA) and docosahexaenoic acid (DHA), overall production of pro-inflammatory molecules decreases while the production of anti-inflammatory molecules increases. In addition to proper rehab practices, omega-3 fatty acid supplementation can reduce the overall inflammatory status and give the joint and its surrounding tissues time to heal. Supplementing your horse with Equine Omega Complete can also prevent joint damage by counteracting the cycle of inflammation before it has time to cause any serious damage.

Proper Cell Development and Function:

Every cell in the body is surrounded by fat. Omega-3 and omega-6 fatty acids compete for incorporation into the cell membrane. When greater quantities of omega-3 fatty acids are present in the diet, they are integrated into the membrane at the expense of omega-6 fatty acids. As part of the cell membrane, omega-3 fatty acids increase fluidity, flexibility, permeability and the activity of enzymes that bind to the membrane, Omega-3 fatty acids have a key function in the formation, growth and regeneration of cells and in the transmission of cell signals throughout life.

Increased Caloric Density:

Fat has 2.25 times as much energy, by weight, as carbohydrate. Feeding fat provides a means to eliminate a portion of the carbohydrate from the diet as a source of calories. Fat is a great energy source for working horses that does not make them hot or overwhelm their system.

An Alternative Energy Source:

Fat serves as an alternative energy source to calories normally provided by non-structural carbohydrate. Working horses with nutritional diseases, such as Equine Metabolic Syndrome or insulin resistance still need a fuel source beyond what their hay provides. Offering fat as an alternative calorie source reduces the digestive and metabolic risks that are often correlated with diets high in starch and sugar.

Reduced Stress, Anxiety and Depression:

Supplementation with fat in an oil form can positively modify behavior when used to replace some of the non-structural carbohydrates in the diet. Inadequate fat intake contributes to unstable blood sugar patterns that stress the horse's metabolism by causing an increase in the release of the hormones cortisol, adrenalin and insulin. Increased hormone release affects mood, performance, immune function and injury prevention. Since fats are digested more slowly, blood sugar levels are maintained, reducing the amount and frequency of stress hormones.

Enhanced Athletic Performance:

The accumulation of lactic acid in the muscle leads to muscle fatigue and soreness. Horses utilizing fat as an energy source for long-term work often show increased stamina and tend to recover faster after work. During hard workouts, glycogen stores in the muscle and liver are also used, Supplemented fat can be used as an alternative, sustainable energy source, saving stored glycogen.

Increased Blood and Oxygen Flow:

Omega-3 fatty acids increase the flexibility of red blood cell membranes. Flexible cell membranes aid in efficiency of blood flow and delivery of oxygen during a hard workout. When horses exercise heart rate and the density of the cells increase and blood becomes more viscous, making the flexibility of red blood cells that much more vital for proper oxygen delivery.

A Healthy Digestive System:

Adding fat to the diet allows for smaller meal size without reducing the amount of calories. This lowers the risk of digestive disorders by allowing the concentrate portion of the meal to be fully digested before it passes into the hindgut. Improperly or partially digested concentrates can ferment in the hindgut, disturbing the delicately balanced population of bacteria. Feeding fat is also thought to help reduce the incidence and severity of gastric ulcers. Supplementation of the omega-6 fatty acids, linoleic acid and arachidonic acid promote the production of mucus, which acts to protect the gastric lining from corrosive acid.

Insulin Regulation:

Equine diets with added polyunsaturated fats increase insulin sensitivity when compared to diets high in starch and sugar. Omega-6 and omega-3 polyunsaturated fatty acids have been shown to influence the ability of insulin to recognize and bind cell membranes, affectively impacting glucose production and transport.

A Strong Immune System:

Omega-6 fatty acids are pro-inflammatory and are beneficial during infection and sickness. Omega-6's promote blood clotting inflammation and immune system responses, however if these processes get carried away, they can actually become more harmful than helpful. The diet must therefore contain the correct proportion of omega-6 to omega-3 fatty acids in order to keep these processes in check,

A Healthy Respiratory System:

Constantly exposed to allergens like dust, mold, pollen and air pollution, the horse's sensitive respiratory system benefits from low-dust, omega-3 fatty acid oil supplementation. Omega-3 fatty acids support lung function in horses and are thought to help treat exercise-induced pulmonary hemorrhage. Omega-3's are crucial players that determine the flexibility of cell walls and the flexibility of red blood cells is particularly important during exercise, increased elasticity of red blood cells allows easier passage through narrow blood vessels in the lungs, ensuring adequate delivery of oxygen.

A healthy reproductive system:

Supplementation with omega-3 fatty acids enhances reproductive function in both stallions and mares. Adding omega-3 fatty acids to the diet of breeding stallions can increase daily sperm output, the percentage of morphologically normal sperm and sperm motility. In breeding mares, omega-3 fatty acid supplementation may influence fertility and the ability of the mare to maintain pregnancy.

Healthy Skin and Hair Coat:

Fundamental nutrients support normal skin and coat development and function. An essential fatty acid deficiency may cause dry coat, scaly skin or hair loss. Essential omega-6 and omega-3 fatty acids are building blocks that promote the health of skin and hair cells. With the addition of fat to the diet there may be a greater amount of oil secreted from glands in the skin, yielding higher oil content in the hair and providing a healthy looking shine.

Strong, Healthy Hooves:

Amino acids, the building blocks of protein, are key ingredients to promote the growth of healthy hooves. If protein requirements are not met, hoof quality and growth will be compromised. Fatty acids also play a role by protecting the outside of the hoof from moisture damage. Present in correct amounts, they work to seal moisture into the deeper hoof structures and seal out water.

Decreased Free Radical Exposure:

Antioxidants, including many vitamins and minerals, act to protect your horse from reactive oxygen species, or free radicals. Vitamin E is a strong antioxidant that works to protect the integrity of the cell membrane by trapping reactive oxygen species during states of oxidative stress. Muscle, nerve and immune systems also rely on antioxidants like vitamin F in order to function properly. Fatty acids, particularly unsaturated fatty acids, are prone to oxidation. Addition of vitamin E to unsaturated fatty acid supplements reduces the risk of rancidity and increases shelf life. Vitamin E does not appear to be toxic to horses even at relatively high intakes. No upper threshold has been recorded.

Thermal Load Regulation:

In order to regulate and maintain normal body temperature, the horse must dissipate heat made during the digestion and absorption of nutrients. Fat has a lower thermal load than other calorie sources. Feeding fat may help reduce the thermal load of a working horse. This may be especially helpful for horses that live and work in hotter climates,

Although the present invention has been described and illustrated with respect to a preferred embodiment and a preferred use thereof, it is not to be so limited since modifications and changes can be made therein which are within the full intended scope of the invention.

I claim;:

1. A supplement composition comprising: cranberry oil in the volume range of 10-30%, 20-40% soybean oil in the volume range of and fish oil in the volume range of 30-70%.

2. The composition of claim 1 comprising cranberry oil in the volume range of 20-30%.

3. The composition of claim 2 comprising 20% cranberry oil.

4. The composition of claim 3 further comprising 25% soybean oil and 55% fish oil.

5. The composition of claim 1 wherein the cranberry oil is extracted from cranberry seeds.

6. The composition of claim 5 wherein the cranberry oil is extracted from the cranberry seeds via cold expeller extraction.

7. An equine nutritional supplement consisting essentially of: cranberry oil in the volume range of 10-30%, 20-40% soybean oil in the volume range of and fish oil in the volume range of 30-70%.

8. The composition of claim 6 consisting essentially of cranberry oil in the volume range of 20-30%.

9. The composition of claim 8 consisting essentially of 20% cranberry oil.

10. The composition of claim 9 further consisting essentially of 25% soybean oil and 55% fish oil.

11. The composition of claim 7 wherein the cranberry oil is extracted from cranberry seeds.

12. The composition of claim 12 wherein the cranberry oil is extracted from the cranberry seeds via cold expeller extraction.

13. An animal nutritional supplement consisting of: cranberry oil in the volume range of 10-30%, 20-40% soybean oil in the volume range of and fish oil in the volume range of 30-70%.

14. The composition of claim 13 consisting of cranberry oil in the volume range of 20-30%.

15. The composition of claim 14 consisting of 20% cranberry oil.

16. The composition of claim 15 further consisting of 25% soybean oil and 55% fish oil.

17. The composition of claim 13 wherein the cranberry oil is extracted from cranberry seeds.

18. The composition of claim 17 wherein the cranberry oil is extracted from the cranberry seeds via cold expeller extraction.

19. A method of treating an illness in an animal, comprising the steps of providing the animal a supplement composition comprising: cranberry oil in the volume range of 10-30%, 20-40% soybean oil in the volume range of and fish oil in the volume range of 30-70.

20. The method of claim 19 wherein the animal is selected from the group consisting of an equine, a canine or a feline.

21. The method of claim 20 wherein the illness is selected from the group consisting of cancer, Cushing's disease or anihydrosis.

22. The method of claim 2 wherein the animal is an equine and the illnesses is selected from the group consisting of Cushing's disease or anihydrosis.

23. The method of claim 21 wherein the animal is a canine and the illness is cancer.

24. The method of claim 23 wherein the cancer is hemangiosarcoma.
